(19) United States
(12) Patent Application Publication
(10) Pub. No.: US 2025/0213126 A1
(43) Pub. Date: Jul. 3, 2025

(54) HIGH-SPEED LASER SPECKLE CONTRAST IMAGING

(71) Applicant: Aarhus Universitet, Aarhus C (DK)

(72) Inventors: Dmitry Postnov, Aarhus C (DK); Alberto Gonzalez Olmos, Aarhus C (DK)

(73) Assignee: Aarhus Universitet, Aarhus C (DK)

(21) Appl. No.: 18/866,228

(22) PCT Filed: May 17, 2023
(86) PCT No.: PCT/EP2023/063353

§ 371 Date: Nov. 15, 2024

(30) Foreign Application Priority Data
May 17, 2022 (EP) 22173808.1

(51) Int. Cl. A61B 5/021; A61B 5/00; A61B 5/02

(52) U.S. Cl. CPC A61B 5/02125; A61B 5/0077; A61B 5/02007

(57) ABSTRACT

A high-speed laser speckle contrast imaging system is for characterizing pressure wave or pulse wave propagation or vascular conducted response in at least one vessel of a biological target. The apparatus includes: a laser source generating a laser radiation; a high-speed camera configured for capturing at least 1000 frames per second (fps), preferably at least 5000 fps, more preferably 6000 fps of the target; an optical sub-system configured for 1) guiding the laser radiation from the laser source to the target 2) and for collecting and guiding a back-scattered light from the target to the camera; and a processing unit configured for receiving and processing raw image data from the camera for calculating at least one feature related to the pressure wave propagation or vascular conducted response.

### CROSS-REFERENCE TO RELATED APPLICATIONS

The present application is the National Phase entry of International Patent Application No. PCT/EP2023/063353, filed May 17, 2023, which claims priority to European Patent Application No. 22173808.1, filed May 17, 2022, the entire contents of both are hereby incorporated by reference into this application.

### TECHNICAL FIELD

The present disclosure relates to a High-speed laser speckle contrast imaging system and method for characterizing pressure wave or pulse wave propagation and/or vascular conducted response in at least one vessel of a biological target.

### BACKGROUND

Laser speckle contrast imaging (LSCI) is a known technique that provides images with spatial resolution and temporal resolution. LSCI is rather simple and cost efficient. A typical application of LSCI is measurement of microcirculatory blood flow index, for example in the brain, in the skin, and in the kidneys. LSCI based measurements may be performed on alert or anaesthetized patients and LSCI may be applied to the human or animal body.

A technology that is related to LSCI is MESI, or Multiple Exposure Laser Speckle Contrast Imaging. Both LSCI and MESI are based on calculating contrast frames from images taken by a camera. In practice, in both techniques, a light source is employed to illuminate a target, which could be a part of a human or animal body, and a camera is used to take images from the back-scattered light from the target.

Both LSCI and MESI suffer from technological limitations that make them unsuitable for specific characterization of a target. There is a need to overcome these limitations. In particular, prior art LSCI and MESI techniques have a temporal resolution of ˜200 flow frames per second and ˜10 flow frames per second, respectively, becoming a limiting factor when a precise determination of the health of circulatory vessels of a patient is required.

### SUMMARY

Stiffness of vessels in a human body, in particular stiffness of microcirculatory vessels in a retina of a human eye is a very important biomarker that the physician may use for assessing the status of health of a subject. Stiffness of vessels in animals may also be important for research. Stiffness may be extracted by measurement of pulse wave velocity. Pulse wave velocity in a vessel is not the velocity of the fluid or the particles, for example red blood particles, that flow within the vessels. Pulse wave velocity in a vessel is a measure of the velocity of a pressure wave in the vessel.

Pulse wave velocity in microcirculatory vessels of a retina of a human or animal eye is related to pressure wave propagation in the vessel. Pulse wave velocity in microcirculatory vessels of a retina of a human or animal eye is difficult to measure because it is fast, typically within the range of 5 m/s to 14 m/s and observable distances in microcirculatory vessels are short, typically less than a 0.01 m, more typically around 0.001 m. Vessel stiffness can be extracted directly from pulse wave velocity. Current techniques, based on traditional or conventional Laser Speckle Contrast Imaging (LSCI) or Multiple Exposure Laser Speckle Contrast Imaging (MESI) are not capable to address such a fast-changing response like pulse wave velocity in microcirculatory vessels.

Another fast-changing response that LSCI and MESI are not capable to capture is the so called vascular conducted response, which is a response related to a reaction of a vessel to an electrical stimulus, for example an electrical stimulus at a given frequency. The propagation of the electrical stimulus in the vessel, and in particular the vascular conducted response, is important for research and may also be a biomarker indicating the status of health of a human or an animal.

Traditional or conventional LSCI and MESI are currently not capable of measuring stiffness nor vascular conducted response in microcirculatory vessels. Given the limitations of traditional or conventional LSCI and MESI to capture fast responses, the inventors have realized that pulse wave velocity, stiffness, and/or vascular conducted response in microcirculatory vessels can be extracted if high-speed camera is used for the image acquisition.

The present disclosure therefore in a first embodiment relates to a high-speed laser speckle contrast imaging system for characterizing pressure wave propagation or vascular conducted response in at least one vessel of a biological target, the apparatus comprising:
a laser source for generating laser radiation;
a high-speed camera configured for capturing at least 1000 frames per second (fps) of the target, preferably at least 5000 fps, more preferably 6000 fps of the target;
an optical system configured for 1) guiding the laser radiation from the laser source to the target 2) and for collecting and guiding a back-scattered light from the target to the camera, and
a processing unit configured for receiving and processing raw image data from the camera for calculating at least one feature related to pressure wave propagation and/or vascular conducted response in said vessel(s).

The present disclosure further relates to a high speed laser speckle contrast imaging system for characterizing pressure wave propagation or pulse wave velocity of one vessel of a biological target, the apparatus comprising:
a laser source for generating laser radiation;
a high-speed camera configured for capturing at least 1000 frames per second (fps);
an optical sub-system configured for 1) guiding the laser radiation from the laser source to the target 2) and for collecting and guiding a back-scattered light from the target to the camera; and
a processing unit configured for receiving and processing raw image data from the camera for calculating the pressure wave propagation or pulse wave velocity over a length of said vessel.

One advantage of the high speed camera of the presently disclosed approach is that very small dimensions and/or high pulse wave velocities can be characterized. In particular it is possible to characterize a single vessel, because the high speed camera makes it possible to calculate the pressure wave propagation or pulse wave velocity in a length of the vessel of less than 1 mm (with at least 1000 fps), or less than 0.2 mm (with at least 5000 fps) or less than 0.16 mm (with at least 6000 fps), assuming a pulse wave velocity of at least 1 m/s. Another advantage is the capability of measuring high pulse wave velocities. For example, assuming 1 mm long vessel it is possible to calculate a pulse wave velocity of more than 2 m/s (with at least 2000 fps), or more than 5 m/s (with at least 5000 fps) or more than 6 m/s (for more than 6000 fps). Assuming that the frames per second of the camera are fixed, the minimum length of the vessel to be measured will depend on the maximum pulse wave velocity that needs to be observed. The higher the calculated pulse wave velocities, the higher the required length of the vessel. The advantage of the presently disclosed approach with a high speed camera is that it enables calculation of higher pulse wave velocities or a shorter length of the vessel. Said length of the vessel can for example be determined by simple image processing, for example by counting pixels and by knowing the size of the pixels and the magnification of the image.

The presently disclosed high-speed laser speckle contrast imaging system is built on improvements and modifications of already existent LSCI apparatuses, and may be named HS-LSCI which stands for High-speed Laser Speckle Contrast Imaging. In the present disclosure, HS-LSCI system refers to the presently disclosed high-speed laser speckle contrast imaging system, whereas LSCI refers to existing traditional/conventional laser speckle contrast imaging systems.

In one embodiment of the present disclosure, frames of the camera may be used for calculating spatial contrast frames, and spatial contrast frames may have a same speed as the camera expressed in frames per second.

The presently disclosed system comprises a laser source irradiating a target, low loss optics to drive the radiation to a target and collect a back-scattered light from the target to a camera which is taking pictures at a given high frame rate. A processing unit may be configured to process the raw image data to obtain contrast frames and calculate several biomarkers, including pulse wave velocity, stiffness and vascular conducted response features, such as speed of propagation or diameter change.

The present inventors have realized that, in order to capture the dynamics of a fast-changing response, such as a pulse wave velocity or a vascular conducted response in a microcirculatory vessel, of for example a retina of a human eye, a very high frame rate of a camera is needed, in particular a frame rate of more than 1000 frames per second, preferably more than 5000 fps, even more preferably 6000 fps, or more.

The inventors have realized that, in order to measure a pulse wave velocity of up to 14 m/s for a microcirculatory vessel of about 3 mm in length, the speed of the camera of the presently disclosed apparatus may advantageously be up to 6000 fps. A length of a vessel, a pulse wave velocity of the vessel and a frame rate of the camera may be related to each other. One can think of it as f>v/l, where f is the framerate, v is the velocity and l is the distance. Meaning that framerate should be higher than the relation between v/l, preferably 2 or more times higher, as it increases accuracy. For example, the presently disclosed system may be suitable for measuring a pulse wave velocity of up to 7 m/s for a 1.5 mm long vessel with a 6000 fps camera.

For a given pulse wave velocity of 1 m/s and a vessel of 1 mm, it is required at least a 1000 fps camera. For a given pulse wave velocity of 1 m/s and a vessel of 0.2 mm, it is required at least a 5000 fps camera. For a given pulse wave velocity of 1 m/s and a vessel of 0.16 mm, it is required at least a 6000 fps camera. Vessels of such length can be found for example on microcirculatory vessel in a retina of a mouse eye. It is understood by vessels in the present disclosure as individual vessel segments and not an integration over several vessels, a path of connected vessels or vessels comprised within a laser illuminated area.

The small length of the mentioned vessels sets a spatial limitation of prior art pulse wave velocity as cameras comprising at least 1000 flow fps are required. By flow fps it is understood acquired frames by the camera that can be used to extract at least a property of the vessel. It is known that prior art speckle imaging techniques, such as MESI or DLSI use high fps cameras for data collection, but the processed flow frames have significantly reduced rate.

The present disclosure further relates to a high-speed laser speckle contrast imaging method for characterizing pressure wave propagation or a vascular conducted response in at least one vessel of a biological target, the method comprising the steps of:
irradiating the target by laser radiation;
capturing at least 1000 frames per second (fps), preferably at least 5000 fps, more preferably 6000 fps of the target by a high-speed camera;
calculating at least one feature related to the pressure wave propagation or the vascular conducted response, based on raw image data from the camera, using a processing unit.

The present disclosure further relates to a high-speed laser speckle contrast imaging method for characterizing pressure wave or pulse wave velocity in a vessel of a biological target, the method comprising the steps of:
irradiating the target by laser radiation;
capturing at least 1000 frames per second (fps), preferably at least 5000 fps, more preferably at least 6000 fps, of the target by a high-speed camera;
calculating the pressure wave propagation or pulse wave propagation over a length of said vessel, based on raw image data from the camera, using a processing unit.

It is to be understood that the presently disclosed systems may be configured to implement all the steps of the presently disclosed methods. In particular the processing unit of the presently disclosed system may be configured to implement all the steps directed to the calculation of pulse wave velocity, stiffness and/or vascular conducted response or other features/biomarkers from the raw image data captured by the camera of the presently disclosed system.

### BRIEF DESCRIPTION OF THE DRAWINGS

The present disclosure will in the following be described with reference to the accompanying drawings:

FIG. 1 shows a schematic view of one embodiment of the presently disclosed high-speed laser speckle contrast imaging system;

FIG. 2A-2B show schematic views of respectively theoretical and measured absolute sensitivity change as a function of exposure time for different vessel sizes;
FIG. 3 shows a schematic view of the signal-to-noise ratio SNR as a function of the exposure time, for a blood flow in a vessel of 50 micrometres in lumen diameter;

FIG. 4 shows a schematic view of the normalized variation (speckle noise) as a function of speckle to pixel size ratio according to traditional/conventional LSCI theory (401) and one of discoveries that HS-LSCI system is based on (402);

FIG. 5 shows a flow chart of summarized steps of one embodiment of the presently disclosed method;

FIG. 6 shows a flow chart of main steps of the calculation (600) in one embodiment of the presently disclosed method;

FIG. 7A shows a flow chart of a main step Quantification 1 (602) of the calculation (600) in one embodiment of the presently disclosed method;

FIG. 7B shows a schematic view of an example of raw image data used in one embodiment of the presently disclosed method;

FIG. 7C shows a schematic view of an example of spatial contrast frames in one embodiment of the presently disclosed method;

FIG. 8 shows a flow chart of a main step Enhancement 1 (603) of the calculation (600) in one embodiment of the presently disclosed method;

FIG. 9 shows a flow chart of a main step Enhancement 2 (604) of the calculation (600) in one embodiment of the presently disclosed method;

FIG. 10A shows a schematic diagram of an example of time series with identified peaks, according to one embodiment of the presently disclosed method;

FIG. 10B shows a schematic diagram of an example of time-stamp based average contrast frames, according to one embodiment of the presently disclosed method;

FIG. 11 shows a flow chart of a main step Segmentation (606) of the calculation (600) in one embodiment of the presently disclosed method;

FIG. 12A shows a schematic diagram of an example of temporal contrast frames, according to one embodiment of the presently disclosed method;

FIG. 12B shows a schematic diagram of an example of vessel masks, according to one embodiment of the presently disclosed method;

FIG. 12C shows a schematic diagram of an example of a final vessel mask, according to one embodiment of the presently disclosed method;

FIG. 13 shows a flow chart of a main step Quantification 2 (606) of the calculation (600) in one embodiment of the presently disclosed method;

FIG. 14A shows a schematic diagram of an example of dynamic scattering component, according to one embodiment of the presently disclosed method;

FIG. 14B shows a schematic diagram of an example of dynamics regime, according to one embodiment of the presently disclosed method;

FIG. 15A shows a flow chart of a main step Enhancement 3 (605) of the calculation (600) in one embodiment of the presently disclosed method;

FIG. 15B shows schematic diagrams of examples of low-noise time-stamp based average blood flow index frames, corresponding to different phases of a heart beat, according to one embodiment of the presently disclosed method;

FIG. 16A shows a flow chart of some of the steps of a main step of Extraction (608) of the calculation (600), according to one embodiment of the presently disclosed method;

FIG. 16B shows a flow chart of some further steps of a main step of Extraction (608) of the calculation (600), according to one embodiment of the presently disclosed method;

FIG. 17A shows a schematic diagram of an example of pulse wave delay, according to one embodiment of the presently disclosed method;

FIG. 17B shows a schematic diagram of an example of stiffness, according to one embodiment of the presently disclosed method;

FIG. 17C shows a schematic diagram of an example of pulsatility index, diameter, speed and flow, according to one embodiment of the presently disclosed method;

FIG. 18 shows a schematic diagram of a foot-to-foot pulse delay wave delay measurement performed on a cerebral artery of a control mouse;

FIG. 19A shows a graph of pulse wave profiles at the marked locations, from which foot-to-foot pulse wave velocity can be measured at the positions marked with crosses in FIG. 18; and

FIG. 19B shows a graph of pulse wave profiles at the marked locations, from which foot-to-foot pulse wave velocity can be measured in a cerebral artery of a dementia-diagnosed mouse.

### DETAILED DESCRIPTION

### System

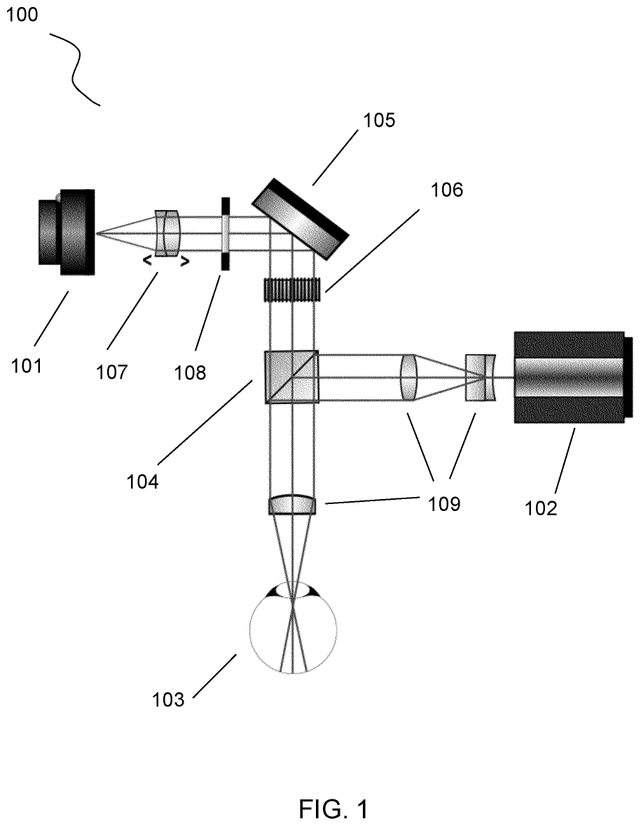

FIG. 1 shows an embodiment of the presently disclosed high-speed laser speckle contrast imaging system, HS-LSCI system.

In one embodiment, the presently disclosed HS-LSCI system may comprise a near-infra-red laser source (102), which may be a high coherence, polarized, temperature controlled NIR laser, corresponding to a wavelength of, for example, 785 or 1050 nm, or other NIR wavelength, and may be, Volume Holographic Grating (VHG) stabilized. A power output of the laser source may be kept below a maximum permissible exposure at a human cornea/retina.

The presently disclosed HS-LSCI system may comprise collimation optics and objective lenses (109) with near 100% transmission, such as 98% or higher, at a wavelength range between 700 and 1200 nm.

In one embodiment of the presently disclosed HS-LSCI system, light from the laser source (102) may be directed, via a polarizing beam splitter (104), to a target (103) and back-scattered light from the target, may be directed, via the polarizing beam splitter (104) to the camera (101). Between the polarizing beam splitter (104) and the camera the light may traverse a dichroic short-pass mirror (105), an iris (108) and a focusing tube lens (107).

In cases where a non-polarizing beam-splitter or a mirror with a pin-hole is used, a linear polarizer (106) may be included in the system for polarizing. In cases where a polarizing beam-splitter is used, a linear polarizer may not be required.

In one embodiment, the linear polarizer (106), the dichroic short-pass mirror (105), the iris (108) and the focusing tube lens (107) may have 100% transmission or nearly 100% transmission at near-to-infrared wavelengths between 700 nm and 1200 nm.

In this context, nearly 100% transmission, is such that it guarantees that the system, with a chosen camera, can operate below the maximum permissible exposure level for the target.

In one embodiment of the presently disclosed HS-LSCI system, the polarizing beam splitter (104) may be configured to convey the light or radiation from the laser source to the target and to convey the back-scattered light or radiation from the target to the camera. The polarizing beam splitter can act so thanks to different polarization of the back-scattered light from the target from the polarization of the light from the laser source. The polarizing beam splitter (104) may be optimized for 700-1200 nm, it may reflect light or radiation from the laser to the eye, and may allow light scattered from the eye, and thus having different polarization, to go through to the camera.

In one embodiment of the present disclosure, the linear polarizer (106) may remove reflected, non-scattered light, it may have near 100% transmission at 700-1200 nm.

In one embodiment of the present disclosure, the dichroic short-pass mirror (105) may have a cut-off at 650 nm, and may have near 100% effective reflection at 700 nm and higher.

In one embodiment of the present disclosure, an iris (108) may be used to control speckle size.

In one embodiment of the present disclosure, the camera (101) may be a more than 1000 fps camera, preferably a more than 5000 fps camera, more preferably a 6000 fps camera, with pixels larger than 20 micrometers, a quantum efficiency of 70% at a wavelength of 785 nm, and may be a 12-bit camera with a sensitivity of 64000 ISO.

In one embodiment of the present disclosure, the camera (101) may have a determined frame rate per second in the whole field of view, than is typically 1024×1024 pixels, and the camera may be suitable to capture a whole vessel or several vessels in one single frame, and not by scanning.

In one embodiment of the present disclosure the target is a retina of a human eye and the at least one vessel is at least one microcirculatory vessel in a retina of a human eye.

In another embodiment of the present disclosure the target is a retina of an animal eye and the at least one vessel is at least one microcirculatory vessel in a retina of an animal eye.

In one embodiment of the present disclosure, the processing unit is configured to, based on raw image data from the camera, calculate a pulse wave velocity in the at least one vessel of the target.

In one embodiment of the present disclosure, the processing unit is configured to extract stiffness of at least one vessel in a target, from a pulse wave velocity of the at least one vessel.

In one embodiment of the present disclosure, the processing unit is configured to, based on raw image data from the camera, calculate a vascular conducted response features, such as velocity or diameter change, of at least one vessel in a target.

In one embodiment of the present disclosure, the laser source is a Near-Infra-Red (NIR) laser source. It may be a high coherence, polarized, temperature controlled NIR laser, at a wavelength of, for example, 785 or 1050 nm and it may be, for example VHG stabilized.

In one embodiment of this disclosure, a power output of the laser source may be calculated to be such that the exposure to the laser radiation to a human eye may be below a maximum permissible exposure at the human cornea/retina.

In one embodiment of the present disclosure, the at least one vessel are microcirculatory blood vessels of diameter size less than 100 micrometers, preferably less than 80 micrometers, and more than 20 micrometers, in a retina of a human or animal eye, or non-retinal vessel.

The frame-rate of the camera, the length of the vessel and the pulse wave velocity or the vascular conducted response that may be calculated by the presently disclosed system are related to each other. According to one example, the presently disclosed system may measure and calculate a pulse wave velocity up to 7 m/s for a vessel of length of 1.5 mm, with the camera having a frame rate of 6000 fps. The presently disclosed system may measure and calculate a pulse wave velocity up to 14 m/s for a vessel of length 3 mm, with the camera having a frame rate of 6000 fps. It is understood by length of a vessel the distance of the two points used to calculate the pulse wave velocity, measured along the path of the vessel. In order to calculate the pulse wave velocity of a length of a vessel it is usually required to perform pulse wave velocity measurements in at least two points of a vessel, wherein the pulse wave velocity can be calculated as the delay of the pulse between the two points. The length of the vessel l might be the distance along the vessel path between two adjacent nodes. The vessel length between the two measured points might comprise one or more nodes, wherein each node bifurcates in at least two vessels. Pulse wave velocity measurements might also be performed in points of length smaller than the distance between two nodes in a vessel.

In one embodiment of the presently disclosed system, the camera is a high sensitivity CMOS camera, having a sensitivity of at least substantially 16000 ISO, preferably at least 32000 ISO, more preferably at least 64000 ISO and/or at least 50% quantum efficiency, preferably at least 60% quantum efficiency, more preferably at least 70% quantum efficiency at a near infra-red spectrum, and wherein said camera has a pixel size of at least 5 micrometres, preferably at least 20 micrometers, and wherein said camera has a near to zero delay between frames, that is the delay is negligible compared to an exposure time of the camera

The inventors have realized that, by having a speed of the camera of up to 6000 fps, an amount of light required, relative to exposure time, by the camera is lower than for traditional LSCI. The inventors have realized that, with exposure time going from long to shorter, that is with frame rates increasing in HS-LSCI as compared to traditional LSCI, the speckle intensity distribution for a specific decorrelation time may be closer to exponential, and not Gaussian as in traditional LSCI, which offsets the optimal average intensity observed on the camera to lower values of the intensity of light. This is advantageous especially in the application of the presently disclosed system to a retina of a human eye, which is sensitive to light exposure. A retina of a human eye may be exposed to a laser radiation below a certain maximum intensity without receiving damage. For applications related to measurement of biomarkers in vessels of a retina of human eyes, it is therefore advantageous that the presently disclosed system may work at optimal values of intensity which are below the maximum that a human eye may tolerate.

The amount of needed light Y may be a function of exposure time T and correlation time tauc according to the following equation: Y=X(T/tauc)/T, where X is a coefficient that is a function of a relation between exposure time and decorrelation time, wherein the correlation time is an indication of the speed of the particles in the vessel. X becomes smaller for T close to tauc. In practice it means that for shorter exposure times X decreases, thus Y does not grow as fast as the decrease in T.

FIG. 2A-2B show respectively theoretical and experimental absolute sensitivity change as a function of the exposure time. The exposure time is inversely related to the frame rate of the camera, that is, for example, an exposure time of 0.2 ms with a nearly zero delay between frames corresponds to a frame rate of 5000 fps.

The inventors have realized that a higher framerate in HS-LSCI as compared to traditional/conventional LSCI brings several advantages.

FIG. 2A shows theoretical absolute sensitivity change (200) as a function of exposure time for a large vessel (202), that is a vessel of lumen diameter or diameter more than 100 micrometers, a medium vessel (201), that is a vessel with lumen diameter or diameter comprised between 20 and 100 micrometers, and a very thin vessel, that is a vessel with lumen diameter below 20 micrometers (203). It is clear from the curves that an optimum value of absolute sensitivity change for medium vessels, is obtained with an exposure time around 0.2 ms, corresponding to a frame rate of about 5000 fps or higher. Vessels in a retina of a human or animal eye, that can be visualised and bear useful information for diagnostics or research, are typically in the range between 20 to 200 micrometres. (but can be larger or smaller).

The use of a high-speed camera is therefore advantageous in increasing the sensitivity to change of the system, which contributes to the possibility of using less power per frame from the laser source, such that said power is below a maximum permissible irradiation of a retina of a human eye. In addition, a frame rate above 5000 fps contributes to a better signal-to-noise ratio.

In one example, HS-LSCI makes exposure time about 10 times shorter than tradition LSCI, but the total amount of required light increases only by a factor 2 or 3, with a total decrease of amount of light per frame.

FIG. 2B shows experimental absolute sensitivity change as a function of exposure time (210) for a large vessel (212), that is a vessel of lumen diameter or diameter more than 100 micrometers, a medium vessel (211), that is a vessel with lumen diameter or diameter comprised between 20 and 100 micrometers, and a very thin vessel, that is a vessel with lumen diameter below 20 micrometers (213). Also from this figure it is clear that optimum exposure time is around 0.2 milliseconds, or below. Therefore the high frame rate of HS-LSCI brings the benefit of better sensitivity and lower needed irradiation power. Lower irradiation power is particularly useful when the target is a retina of a human eye, because a retina of a human eye may not be exposed to a radiation which is greater than a maximum permissible radiation. Better sensitivity is useful because it permits to generate low noise contrast frames which may be used for calculation of fast responses such as pulse wave velocity or vascular conducted response in vessels of lumen diameter or diameter comprised between 20 and 100 micrometers, and lengths down to 1 mm.

The presently disclosed system may measure stiffness or other biomarkers for vessels with a with variable lengths and variable pulse wave velocities, according to the equation f>v/l, where f is the framerate, v is the velocity and l is the distance. Meaning that framerate f should be higher than the relation between v/l, preferably 2 or more times higher, as it increases accuracy. Using a high framerate camera configured for capturing 1000 frames per second, preferably 5000 frames per second or more preferably 6000 frames per second of the target allows to improve the resolution of the measured pulse wave velocity v in the vessels or to improve the length resolution of the measured vessels, allowing to measured high pulse wave velocity simultaneously on short length vessels. The final pulse wave velocity and length resolutions is determined by the final frames per second used in the camera and according to the equation f>v/l.

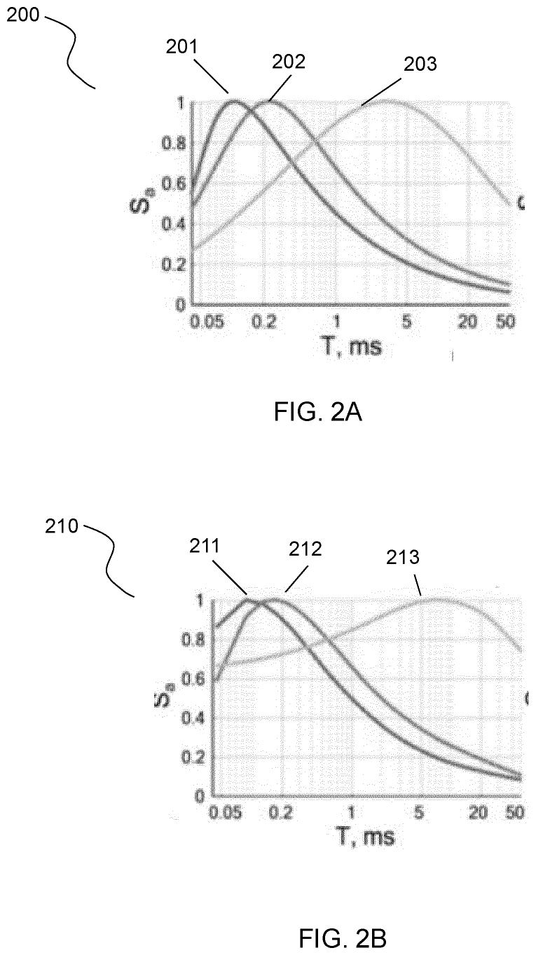

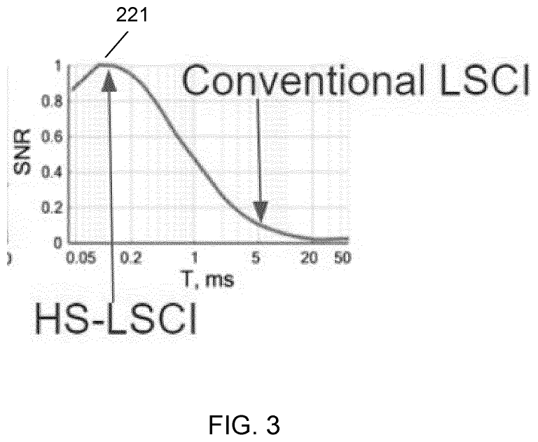

The inventors have further realized that a high frame rate in HS-LSCI as compared to traditional/conventional LSCI brings further advantages. FIG. 3 is showing a signal-to-noise ratio (SNR) as a function of exposure time (221) of the presently disclosed system applied to a vessel of 70 micrometers in diameter. From this figure it is clear that an optimum signal-to-noise ratio is achieved for exposure times around or below 0.2 ms, corresponding to frame rate of the camera of 5000 fps or higher, preferably 6000 fps. Traditional/conventional LSCI has a typical exposure time of 5 ms, which as a lower SNR and cannot achieve measurement/calculation of pulse wave velocity and/or stiffness and/or vascular conducted response for microcirculatory vessels in a retina of a human or animal eye, whereas HS-LSCI benefits from a higher signal-to-noise ratio which is advantageous in, for example, measurement/calculation of pulse wave velocity and/or stiffness and/or vascular conducted response for microcirculatory vessels in a retina of a human or animal eye.

Both optimum sensitivity and optimum SNR achieved with frame rates above 1000 fps, preferably above 5000 fps, more preferably 6000 fps, among other features, contribute to the presently disclosed system being able to measure/calculate pulse wave velocity and/or stiffness and/or vascular conducted response for microcirculatory vessels in a retina of a human or animal eye.

A signal-to-noise ratio (SNR) may be defined by ratio of pulse harmonics power to noise pedestal in a FFT power spectrum of contrast data.

In the presently disclosed HS-LSCI system, the camera has a near-to-zero delay between frames, that is a delay between frames negligible as compared to the duration of a frame. The inventors have realized that such near-to-zero delay between frames is advantageous when averaging frames together, for example for building surrogate frames for multi-exposure times. As the delay between frames is near-to-zero, also less light is needed because the actual frame is extending during almost a full exposure time and light is captured by the camera during almost a full frame period and not during a limited portion of the frame period, which decreases the requirements on the needed amount of light. This means that a light on target may be below a maximum permissible and the camera may still be successfully register useful raw data. A near-to-zero delay between frames is advantageous for high-speed laser speckle contrast imaging in general as it means there is no data lost and the maximum framerate possible for a given exposure time is achieved. A multi-exposure time may comprise a multiple of an exposure time. So, for example a multiple-exposure surrogate frame may be obtained by taking two consecutive frames each with exposure time T, or three, or several, and average them together.

In the one embodiment of the presently disclosed system, the camera may have pixel size such that a speckle to pixel size ratio is below 2, preferably below 1, even more preferably 0.5. That is, the number of speckles per pixels may be more than 0.5, preferably more than 1, more preferably 2.

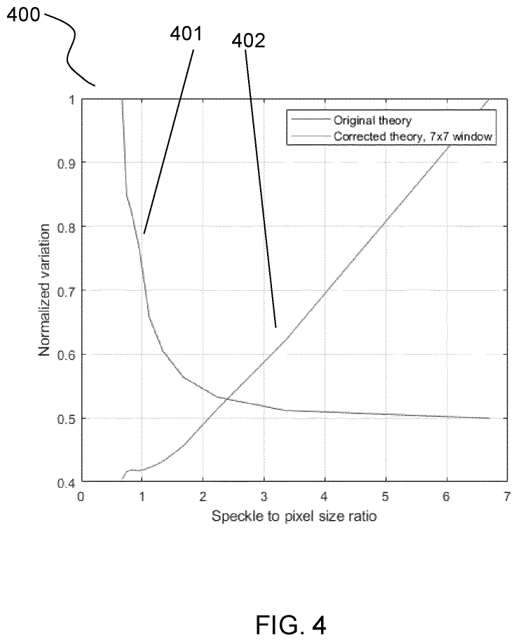

The inventors are aware of the theory behind traditional/conventional LSCI. According to the traditional/conventional theory of LSCI the optimum speckle to pixel size ratio is equal to or more than 2. This is shown in FIG. 4 by curve (401), which is the normalized variation, that is the normalized speckle noise level, as a function of the speckle to pixel size ratio, according to traditional/conventional LSCI theory. The inventors have realized that, in practice, instead, the variation or noise is a function of the speckle to pixel ratio and is defined by the curve (402) in FIG. 4, as it depends on the utilized window size, and therefore has an optimum when the speckle to pixel size ratio is below 2. Window size may typically be 5×5 pixels, or 7×7 pixels. The inventors have realized that, in practice, the variation caused by speckle noise is defined by finite number of speckles within the window used for contrast calculation. For 7×7 pixels window the variation as a function of the speckle to pixel ratio is defined by the curve (402). Such discrepancy is explained by the original theory assuming number of speckles in the window to be large enough to not affect the variation. This, however, is not the case in the practical applications, where 2 speckles per pixel means that there are only 12 speckles within the 7×7 window. It is not practical to increase window size as it decreases spatial resolution, thus the size of the speckles has to be reduced to increase the number of speckles in the window and, as a result, reduce the variance caused by speckle noise. It is noted that in FIG. 4 the noise level is a function of the speckle to pixel size ratio, and not of the speckle to pixel number ratio. A value of 2 in the x-axis of FIG. 4 corresponds to speckle to pixel size ratio of 2, and therefore to a number of 2 pixels per speckle as the speckle is twice as large as a pixel. According to the new theory that the inventors have realized, an optimum speckle to pixel size ratio is below 2. Based on this new theory, the inventors have realized that a greater dimension of the pixel size of the camera is advantageous, with respect to the size of a speckle, and the inventors have chosen a camera with a pixel size of more than 5 micrometers, preferably larger than 20 micrometers, in order to accommodate, for example, one speckle per pixel or more, or preferably 1.5 speckles per pixel or more, or more preferably 2 speckles per pixel or more. Pixel size is defined as a linear size, wherein a pixel is assumed to be square. Therefore a pixel size of 5 micrometers, here indicates a square pixel of 5×5 micrometers. The design choice of the inventors is to have a lower than 2, preferably lower than 1, speckle to pixel size ratio, in order to minimize the speckle noise level, or variation level, and therefore being able to measure and calculate fast responses such as pulse wave velocity and/or stiffness, and or vascular conducted response of a microcirculatory vessel. In the presently disclosed system, given an optimum value of the speckle size of about 20 micrometers or less, a pixel size of 20 micrometers or more gives lower variation noise level as compared to traditional/conventional LSCI.

In one embodiment, the presently disclosed system may comprise a polarizing beam splitter, a mirror with a through-hole or a 9:1 or higher ratio beam splitter. A mirror with a through-hole may also be referred to as a pin-hole mirror. In the presently disclosed system, radiation from the laser source is conveyed to a target, and back-scattered light or radiation from the target is conveyed to the camera. This functionality may be implemented by use of one of the following optical components: a polarizing beam splitter, a pin-hole mirror or a 9:1 or higher beam splitter. Any of these components, in the presently disclosed system, are such that the back-scattered light from the target to the camera is almost not attenuated, that is the transmission is near 100% from the target to the camera. This way, the on-target laser power of the laser source may be kept to a value below a maximum permissible value. In order to minimize the output power of the laser source the optical sub-system of the presently disclosed high-speed laser speckle contrast imaging system may have an at least overall 90% transmission, preferably at least 95% transmission, more preferably nearly 100%, hence the loss of the optical sub-system may be minimized to below 10%, preferably below 5%, most preferably negligible.

### Method: General

It is understood by pressure wave propagation to the velocity at which a pressure wave moves in a fluid, such as blood in the circulatory system. Such waves may be continuous waves of pressure fluctuations. The pressure wave propagation velocity is the velocity at which the pressure wave moves in the fluid and is also referred to as the velocity of sound in that medium. A pressure wave propagation might be generated by an external or internal stimuli such as for instance the propagation of a pressure wave caused by a local dilation or contraction of a vessel. The unit measuring the pressure wave propagation is velocity, such as m/s.

It is understood by pulse wave velocity to the particular pressure wave propagation through a vessel, being the origin of such pulse the heartbeat. The pulse wave velocity comprises distinct pressure peaks. The unit measuring the pressure wave propagation is velocity, such as m/s.

In one embodiment of the presently disclosed method, the laser irradiation on the target may be below 1 mW/cm2, preferably substantially below 0.25 mW/cm2, such that the irradiation is below a maximum permissible.

In one embodiment of the presently disclosed method, the target may be a portion of a retina or a retina of a human or animal eye, and the calculated feature may be a pulse wave velocity and/or a stiffness of at least one vessel in the retina or a vascular conducted response in a vessel.

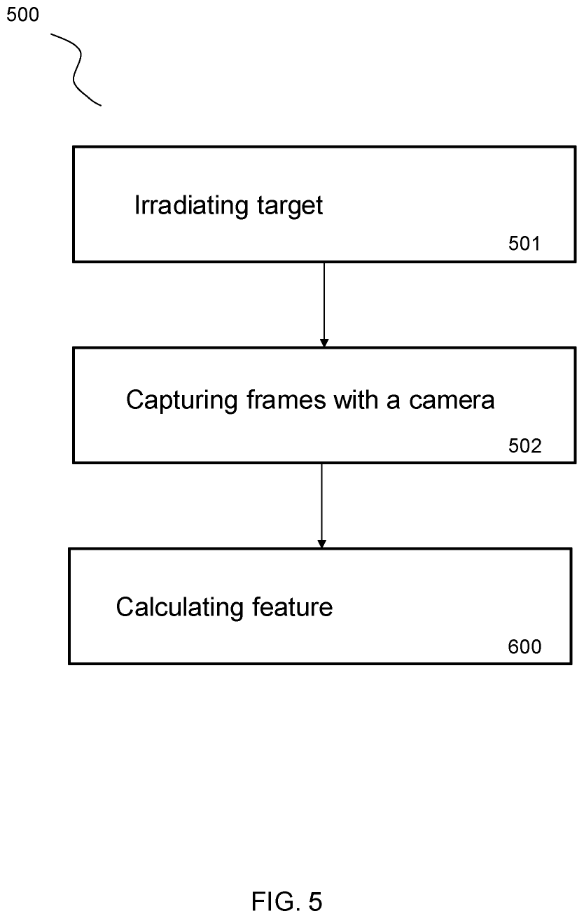

FIG. 5 shows a flow chart of the summarized steps (500) of the presently disclosed method: irradiation of a target (501); capturing frames or raw image data with a camera (502); calculating at least a feature by using a processing unit (600). It is understood that the calculation (600) of the feature by a processing unit, such as a programmable device configured to execute programs and algorithms related to image processing and contrast processing, comprises at least one or several computer-implemented steps.

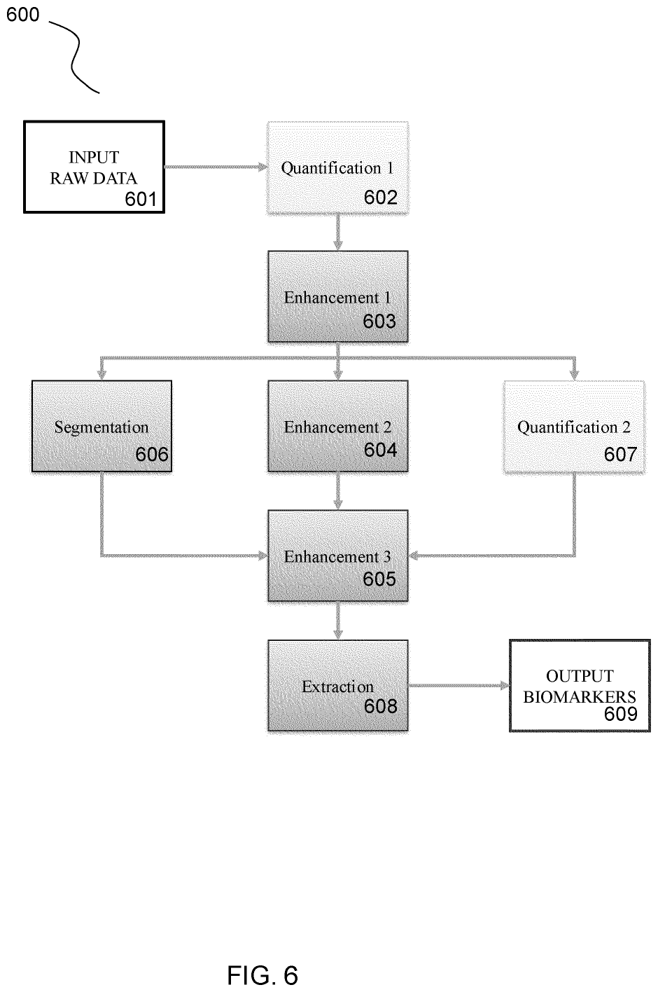

In one embodiment, the calculation (600) may be performed on a processing unit and may comprise one or more main steps. The flow chart in FIG. 6 shows the main steps and their interdependencies. In particular the calculation (600) may comprise main steps of: Input raw image data (601), Quantification 1 (602), Enhancement 1 (603), Enhancement 2 (604), Enhancement 3 (605), Segmentation (606), Quantification 2 (607), Extraction (608) and Output features/biomarkers (609). Each of the above main steps may comprise at least one step.

### Method: Input Raw Image Data

In one embodiment of the presently disclosed method, raw image data is input to the calculation (600) executed on the processing unit of the presently disclosed system.

### Method: Quantification 1

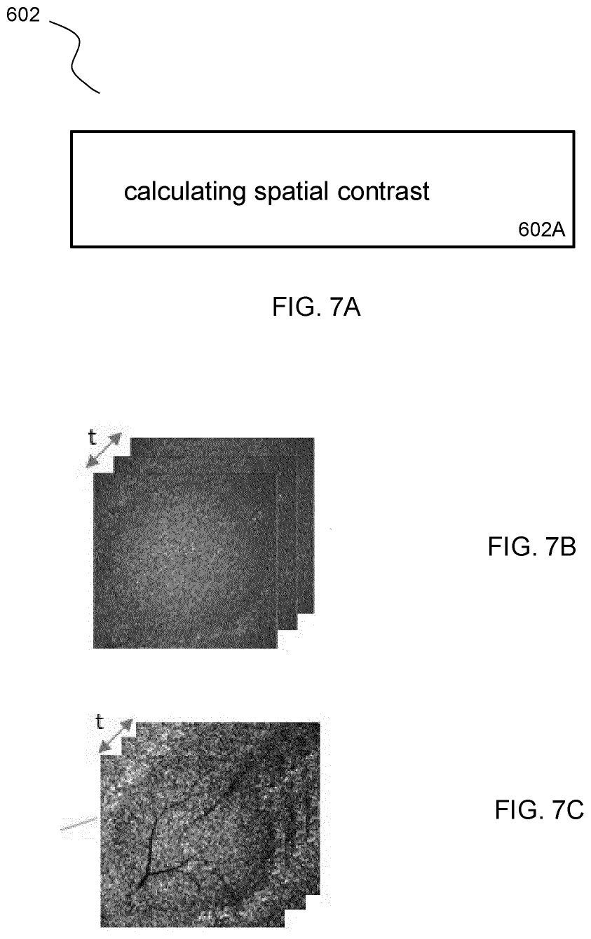

In one embodiment, the calculation (600) of the presently disclosed method may comprise a main step of Quantification 1 (602). The main step of Quantification 1 may comprise the step of, based on raw image data, calculating spatial contrast for each frame of the raw image data and/or calculate spatial contrast frames. FIG. 7A shows a flow chart of the main step Quantification 1 (602), comprising the step of calculating spatial contrast frames from raw image data (602A). In one embodiment of the presently disclosed method, a camera is capturing raw image data at a rate of more than 1000, preferably more than 5000, more preferably 6000 frames per second, and for each raw image a spatial contrast frame is calculated. Raw image data is typically a greyscale image wherein each pixel corresponds to a grey value, which is an integer number from 0 to a maximum depending on the number of bits of the camera, wherein the bits of the camera may be, for example, 12. In the presently disclosed method, neighbouring pixel values of the raw image data are used to calculate a per pixel contrast value, and therefore spatial contrast frames are obtained. In each spatial contrast frame, each pixel is associated with a contrast value, for example a floating point number comprised between 0 and 1, where 1 corresponds to no motion and 0 corresponds to high motion or a very blurred image.

FIG. 7B shows a schematic diagram of an example of raw image data in one embodiment of the presently disclosed method. FIG. 7C shows a schematic diagram of an example of spatial contrast frames in one embodiment of the presently disclosed method, wherein the spatial contrast frames are obtained by raw image data, or registered raw image data.

### Method: Enhancement 1

In one embodiment, the calculation (600) of the presently disclosed method may comprise a main step of enhancement 1, comprising the step of applying motion registration to raw image data.

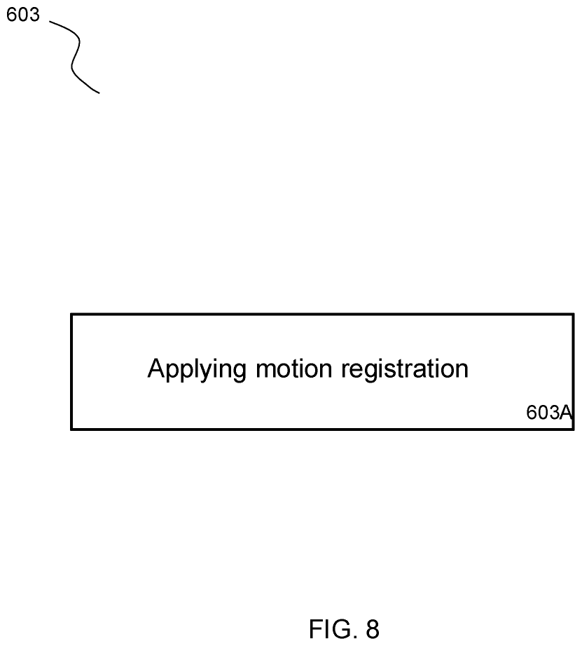

FIG. 8 shows a flow chart of the main step Enhancement 1 (603), comprising the step of applying motion registration (603A). Spatial contrast may be applied directly to raw image data or to registered image data, if motion registration is required.

### Method: Enhancement 2

In one embodiment, the calculation (600) of the presently disclosed method may comprise a main step of Enhancement 2. The main step of Enhancement 2 may comprise the step of calculating accurate time-stamp based average contrast frames by averaging contrast frames belonging to a same time-stamp cycle, such as a same phase of a heart beat.

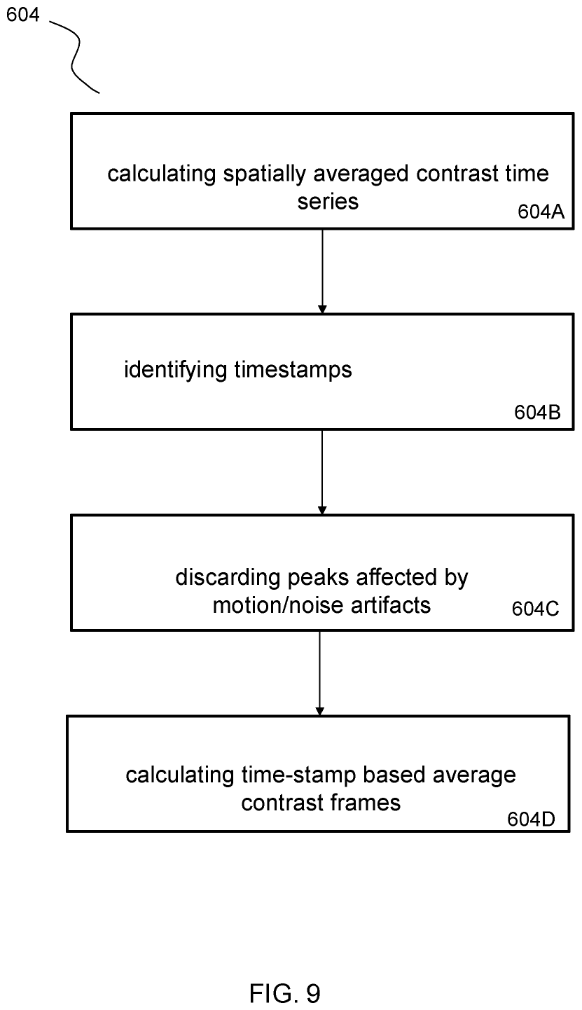

As shown in FIG. 9, in one embodiment of the present disclosure, the main step of Enhancement 2 (604) may comprise the steps of: based on spatial contrast, calculating spatially averaged contrast time series (604A); identifying timestamps of positive and negative peaks of said time series (604B); discarding peaks affected by motion/noise artifacts (604C); and calculating time-stamp based average contrast frames by averaging contrast frames belonging to a same time-stamp cycle, such as a same phase of a heart beat (604D).

In one embodiment of the present disclosure, the main step of Enhancement 2 may comprise the steps of: based on spatial contrast, calculating spatially averaged contrast time series; identifying timestamps of positive and negative peaks of said time series; discarding peaks affected by motion/noise artifacts; and calculating time-stamp based temporal contrast frames from raw data belonging to a same time-stamp cycle, such as a same phase of a heart beat, In this embodiment time-stamp based temporal contrast frames are calculated instead of time-stamp based average contrast frames.

Time-stamp based temporal contrast frames may be used instead of time-stamp average contrast frames achieving better accuracy and for calculation of stiffness of vessels of smaller dimensions.

Time-stamp based temporal contrast frames are calculated on raw data corresponding to a same cycle to obtain, together with optimum temporal resolution, also an optimum spatial resolution, that is same temporal resolution but higher spatial resolution than time-stamp average contrast frames. Using time-stamp based temporal contrast frames in the presently disclosed method, instead of time-stamp average contrast frames provides better spatial resolution which is advantageous when measuring stiffness of vessels of smaller dimensions.

Because spatial contrast frames have a very good temporal resolution, the inventors have realized that spatial contrast frames may be used to extract accurate time series, which represent the dynamics or blood flow as a function of time. Timestamps of positive and negative time series may be identified from the time series, and using these time stamps, cycles may be identified, such as phases of a heart beat. In order to improve signal-to-noise ratio, peaks affected by motion/noise artefacts may be discarded. Cycles may be also identified, in one embodiment, by registering information from an electro-cardio-gram (EEG) of a subject. In one embodiment of the present method, once cycles have been identified, time-stamp based average contrast frames may be calculated by averaging contrast frames belonging to a same time-stamp cycle, such as a same phase of a heart beat. The inventors have realized that, this way the temporal resolution of the contrast may be increased and the performance been enhanced, because contrast frames belonging to a same time-stamp cycle are averaged within each other, reducing the effect of noise.

The inventors have further realized that both temporal and spatial resolution may be very high by calculating contrast based on raw data corresponding to a same time-stamp cycle, such a same phase of a heart beat, obtaining time-stamp based temporal contrast frames from raw data belonging to a same time-stamp cycle, such as a same phase of a heart beat.

In one embodiment, the presently disclosed method may further comprise comprising the steps of: based on spatial contrast, calculating spatially averaged contrast time series; identifying timestamps of positive and negative peaks of said time series; discarding peaks affected by motion/noise artifacts; and calculating time-stamp based temporal contrast frames from raw data belonging to a same time-stamp cycle, such as a same phase of a heart beat, When calculating contrast directly on raw data corresponding to a same cycle, excellent temporal resolution is obtained because contrast calculation is done on collective raw data where the effect of noise is reduced and, at the same time, excellent spatial resolution may be achieved because the contrast is directly calculated on collective raw data, rather than obtained on averaging contrast frames obtained by single frames. This increased spatial resolution gives the possibility to calculate stiffness for even smaller vessels.

The difference between timestamp-based average spatial contrast, conventional temporal contrast and the proposed timestamp-based temporal contrast analysis may be highlighted in the following example. If window of 7×7 pixels is used to calculate spatial contrast frames which are then averaged according to timestamps, then the resulting data has excellent (no loss) temporal resolution of the cycle, but spatial features are blurred according to the size of the window. In practical situations it would mean that the data from vessels which are less than window size in diameter (e.g. less than 7 pixels) would be mixed with the data from surrounding tissue to the point where it is impossible to extract the vessel specific data. On other hand when conventional temporal analysis is used, it utilizes e.g. 25 frames to calculate a single contrast frame. Thus the spatial resolution is preserved, but the temporal resolution is lost across the cycle (e.g. heartbeat). The alternative method proposed by the authors calculates temporal contrast using raw frames belonging to the same phase of the pulse, rather than consecutive frames. This way both spatial and temporal resolutions are preserved without any loss. Timestamp-based temporal contrast method is particularly beneficial for analysing smaller vessels, however it is also more sensitive to motion artifacts, thus is proposed as alternative, but not a replacement for timestamp-based average spatial contrast

In one embodiment of the presently disclosed method, phases of a heart beat may be identified by analysing the peaks of the time series, and/or by correlating with data produced by an electrocardiogram (ECG), the method may further comprise the step of interpolating the time series to scale heart beats and remove heart rate short term variability effects.

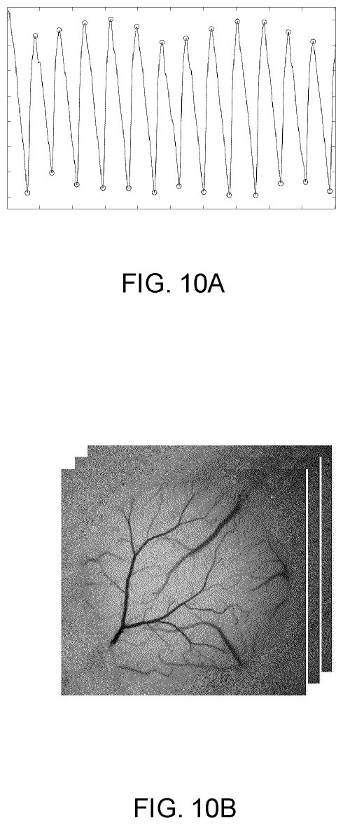

FIG. 10A shows a schematic diagram of an example of time series with identified peaks, according to one embodiment of the presently disclosed method.

FIG. 10B shows a schematic diagram of an example of time-stamp based average contrast frames, according to one embodiment of the presently disclosed method.

### Method: Segmentation

In one embodiment, the calculation (600) of the presently disclosed method may comprise a main step of Segmentation. The main step of Segmentation may comprise the step of obtaining nodes for all vessels in the target by segmentation and skeletonization of temporal contrast frames based on different surrogate exposure times.

In this context, it has to be clear what surrogate exposure times mean. A single exposure frame is a frame captured by the camera in the single exposure time T, which may be, in one example T=0.2 ms. A multi-exposure surrogate frame may be a frame obtained by averaging frames at different multiple exposure times. For example, a multi-exposure surrogate frame may be obtained by averaging a frame taken at time [0, T] ms and a second frame taken at time [T, 2T] ms. In this case the multiple-exposure surrogate time is 2T, as two frames are used for obtaining the multi-exposure surrogate frames. In this case the multi-exposure surrogate frame is obtained by averaging 2 frames. Subsequently frames at time [2T, 3T] and [3T, 4T] may be averaged together to get the next multi-exposure surrogate frame corresponding to a same exposure time of 2T. All possible combinations of a sliding window of 2T may be used for obtaining multi-exposure surrogate frames for exposure time of 2T. 2 or 3 or several consecutive frames may be averaged together in order to obtain multiple-exposure surrogate frames for different surrogate multi-exposure times, for example 2T, 3T, etc. . . .

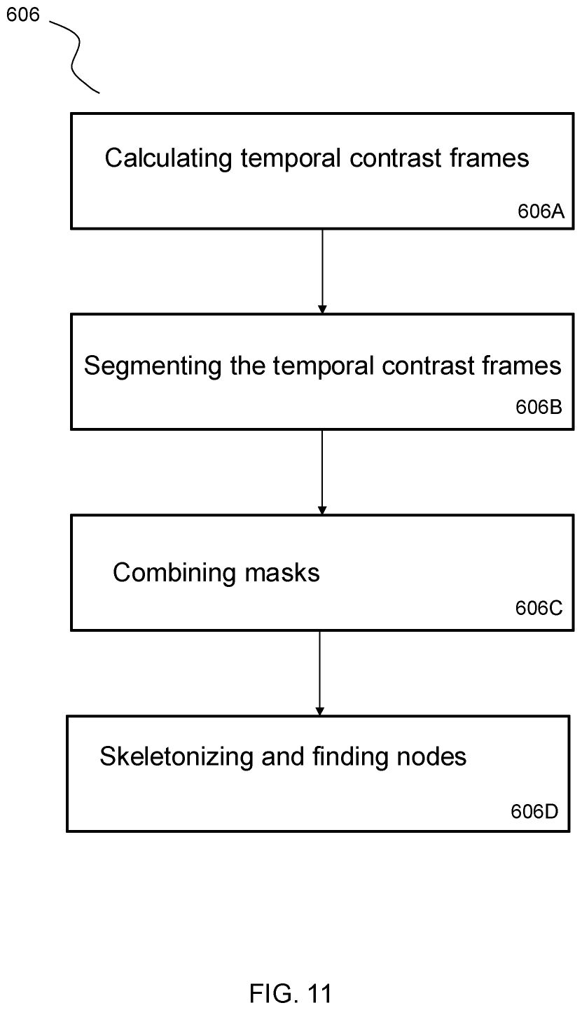

In one embodiment of the present disclosure, as shown in FIG. 11, the main step of Segmentation (606) may comprise the steps of:—) calculating temporal contrast frames over all multi-exposure surrogate frames for different surrogate exposure times (606A);—) segmenting the temporal contrast frames to get vascular structure masks or vessels masks (606B);—) combining masks to create a final mask for the vessels segmentation (606C);—) skeletonizing the final mask (606D); and—) finding nodes for all vessels (606D).

In one embodiment of the present disclosure, for each frame set, multiple-exposure frame sets may be obtained, corresponding to different surrogate multi-exposure times. In one embodiment of the present disclosure, the inventors have realized that an excellent spatial contrast is obtained by calculating temporal contrast among surrogate frames corresponding to a same multi exposure time, for each multi exposure time, generating temporal contrast frames for each multi-exposure time. Vascular structure masks may therefore be extracted from the temporal contrast frames for each multi exposure time and a final mask, with excellent spatial resolution, may be obtained by combining all the masks obtained for different multi-exposure times. Once an excellent resolution final mask is obtained, a successful segmentation and skeletonization may be applied in order to determine accurately the nodes of the vessels.

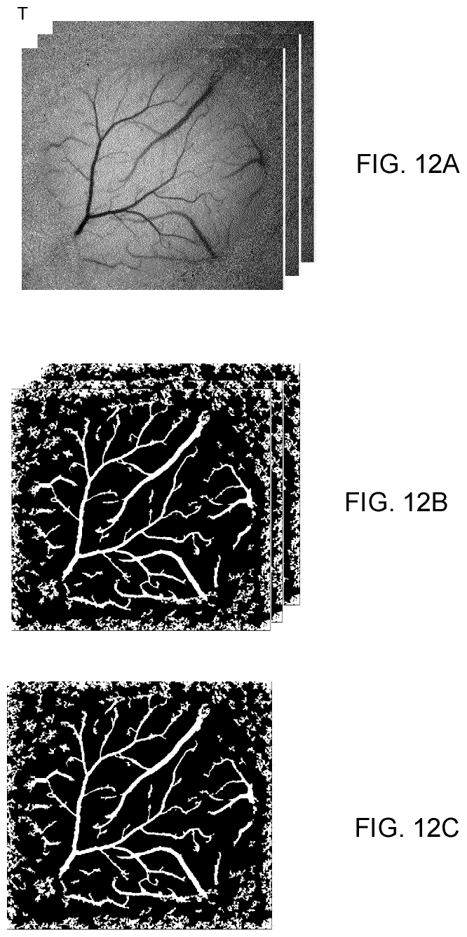

FIG. 12A shows a schematic diagram of an example of temporal contrast frames, according to one embodiment of the presently disclosed method.

FIG. 12B shows a schematic diagram of an example of vessel masks, according to one embodiment of the presently disclosed method.

FIG. 12C shows a schematic diagram of an example of a final vessel mask, according to one embodiment of the presently disclosed method.

### Method: Quantification 2

In one embodiment, the calculation (600) of the presently disclosed method may comprise a main step of Quantification 2. The main step of Quantification 2 may comprise the steps of calculating dynamic (ρ) and/or static scattering component, dynamics regime (n) and offset (C), based on fitting average spatial contrast frames obtained from multi-exposure surrogate frames for different surrogate exposure times with up to 3 light scattering models, and calculating a quantitative blood flow index as a function of time and space coordinates, or a quantitative time-stamp based average blood flow index, using spatial contrast frames or time-stamp based average contrast frames and fitted parameters.

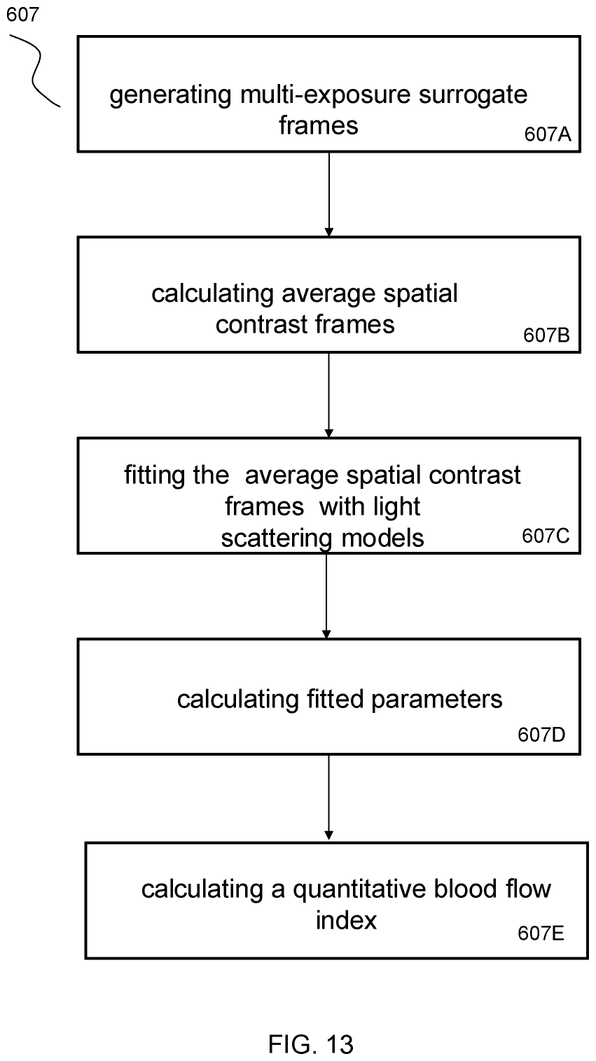

In one embodiment of the present disclosure, as shown in FIG. 13, the main step of Quantification 2 (607) may comprise the steps of: generating multi-exposure surrogate frames from registered raw image data (607A); calculating average spatial contrast frames based on multi-exposure surrogate frames for different surrogate exposure times (607B); fitting the average spatial contrast frames with at least one and up to 3 light scattering models (607C); calculating fitted parameters dynamic and/or static scattering component (ρ), dynamics regime (n) and offset (C) (607D);—) calculating a quantitative blood flow index as a function of time and space coordinates or a quantitative time-stamp based average blood flow index, using spatial contrast frames or time-stamp based average contrast frames and fitted parameters (607E).

In one embodiment of the presently disclosed method, the light scattering models are at least one in the following selection: multiple scattering ordered motion or single scattering unordered motion, multiple scattering unordered motion, single scattering ordered motion.

In one embodiment of the presently disclosed method, multi-exposure surrogate frames are obtained by applying moving average filters of variable length, and each multi-exposure surrogate frame may be obtained from one or more frames of the raw image data or the registered raw image data.

The inventors are familiar with the publication “Choosing a model for laser speckle contrast imaging”, Chang Liu, et al., and the inventors are aware that several light scattering models for laser speckle contrast imaging are available, in particular three separate models. The inventors have realized that fitting the average spatial contrast frames based on multi-exposure surrogate frames for different surrogate exposure times with the three models may have different quality of fitting for each model.

The inventors have realized that fitted parameters, dynamic and/or static scattering component (ρ), dynamics regime (n) and offset (C), are calculated with higher accuracy when up to three separate light scattering models are used to fit the contrast data, such as average spatial contrast frames based on multi-exposure surrogate frames for different surrogate exposure times. As the light scattering models are a function of the exposure time, and as many variables are involved in the equations of the models, the fitting converges when using average spatial contrast frames based on multi-exposure surrogate frames for different surrogate exposure times. In practice, for each multi-exposure time, average spatial contrast is calculated, which means that multiple average spatial contrast are available corresponding to different multi-exposure time. These may be used to fit the equations of the light scattering models to derive the fitted parameters. The fitting of each of the three equations/models may also yield a quality of fitting, and the fitting with the best quality may be used to determine the fitting parameters. This is very advantageous as compared to traditional methods, where only one model is used for the fitting, which may yield poor results in the accuracy of the fitted parameters. Once the fitted parameters are obtained, they may be used for the calculation of a blood flow index or quantitative blood flow index, based on, in one embodiment, spatial contrast frames and the fitted parameters. In one embodiment a time-stamp based blood flow index, or quantitative time-stamp based blood flow index may be calculated based on fitted parameters and time-stamp based average contrast frames.

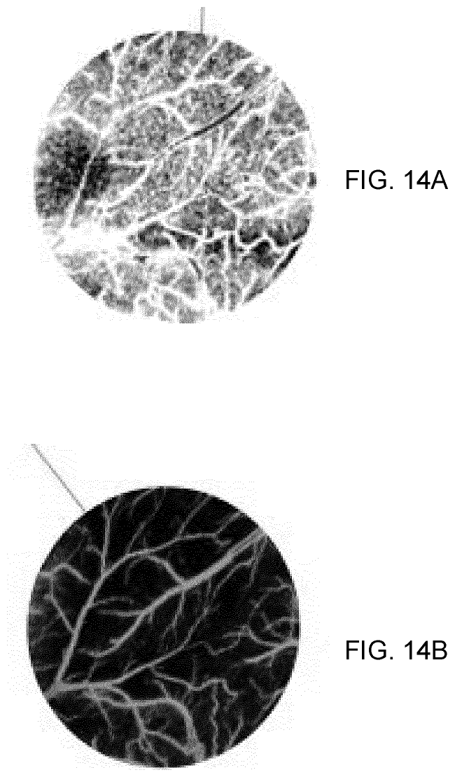

FIG. 14A shows a schematic diagram of an example of dynamic scattering component, according to one embodiment of the presently disclosed method.

FIG. 14B shows a schematic diagram of an example of dynamics regime, according to one embodiment of the presently disclosed method

### Method: Enhancement 3

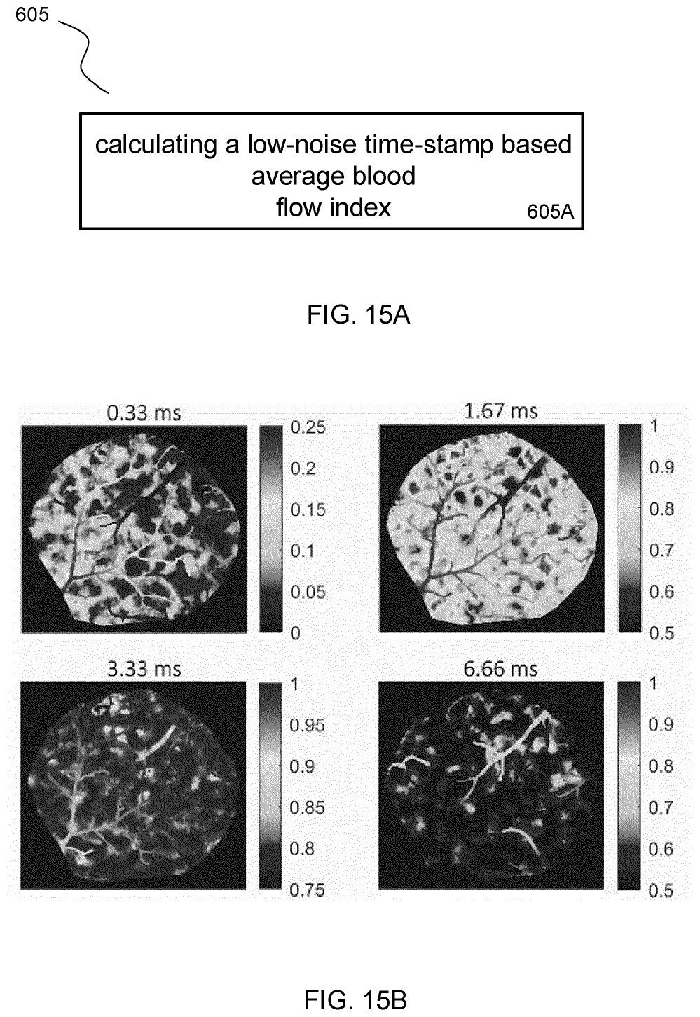

In one embodiment, the calculation (600) of the presently disclosed method may comprise a main step of Enhancement 3. The main step of Enhancement 3 (605), as shown in FIG. 15A, may comprise the steps of: based on quantitative time-stamp based average blood flow index and based on a final vessel mask, calculating a low-noise quantitative time-stamp based average blood flow index obtained by segmentation-based spatial averaging of quantitative time-stamp based average blood flow index such that dynamics of different vessels are not mixed with each other (605A).

In one embodiment, the main step of Enhancement 3 may comprise the steps of: based on time-stamp based average blood flow index and based on a final vessel mask, calculating a low-noise time-stamp based average blood flow index obtained by segmentation-based spatial averaging of time-stamp based average blood flow index such that dynamics of different vessels are not mixed with each other.

The calculations above may be based on quantitative or non quantitative blood flow index. The use of quantitative blood flow index may be advantageous in terms of accuracy of the measured biomarkers and it enables the calculation of some biomarkers.

In particular, use of quantitative blood flow index may result in higher accuracy stiffness measurement, as compared to use of blood flow index that has not undergone quantification step.

The inventors have realized that noise in the blood flow index may be reduced, and therefore accuracy of the blood flow index may be increased, by performing vessel-mask based spatial averaging of time-stamp based average contrast frames. The inventors have realized that, each pixel in each time-stamp based average blood flow index may belong to a vessel, or not, and that only pixels belonging to the same vessel may be used for the vessel-mask based spatial averaging.

It has to be understood that “low-noise” in the expression “low-noise time-stamp average based blood flow index” has to be interpreted as low-noise in comparison with traditional techniques which do not employ the Enhancement 3 main step. In particular, by using the spatial information in the vessel mask, the inventors have realized that, in the calculation of the low-noise quantitative time-stamp based average blood flow index, a pixel within a vessel may be averaged with neighbouring pixels only if the neighbouring pixels belong to the same vessel. This is possible as the vessel mask may be used to extract information about the vessels nodes. This is very advantageous because it ensures that dynamics of different vessels are not mixed together, and that reduces the noise in the low-noise quantitative time-stamp based average blood flow index contributing to making it a “low-noise” blood flow index. Segmentation-based spatial averaging of time-stamp based average blood flow index for calculation of the blood flow index increases therefore the signal-to-noise ratio of blood flow index as dynamics of different vessels are not mixed together.

It is understood that fitted parameters are also used for the calculation of the “low-noise quantitative time-stamp average based blood flow index”

It has to be understood, that the presently disclosed method also may use other steps to reduce the noise of the calculated blood flow index. For example the blood flow index may also be “time-stamp” based, and that means calculated on time-stamp averaged contrast frames, that is contrast frames averaged among other contrast frames belonging to a same time-stamp cycle, such as a same phase of a heartbeat.

### Method: Extraction

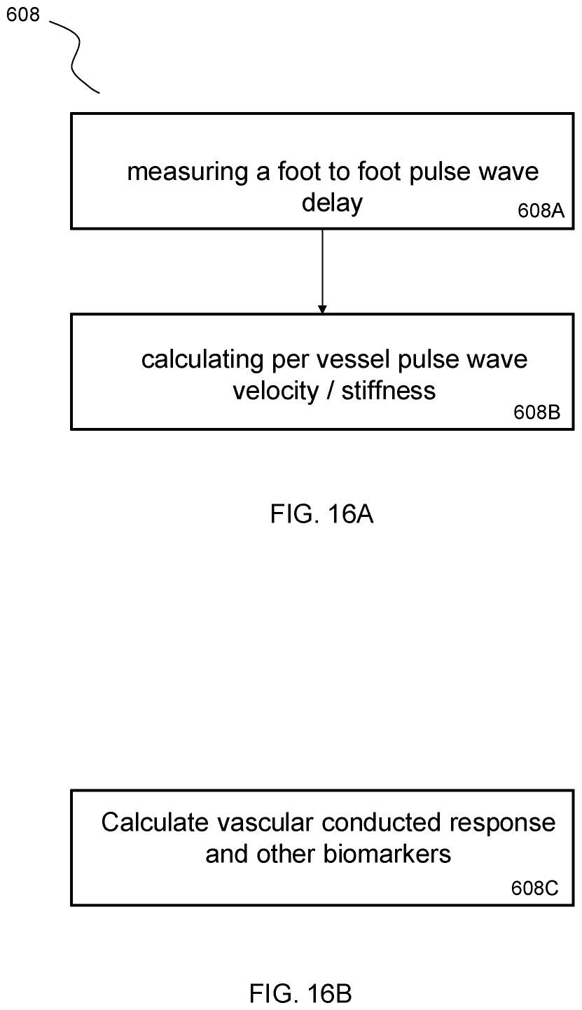

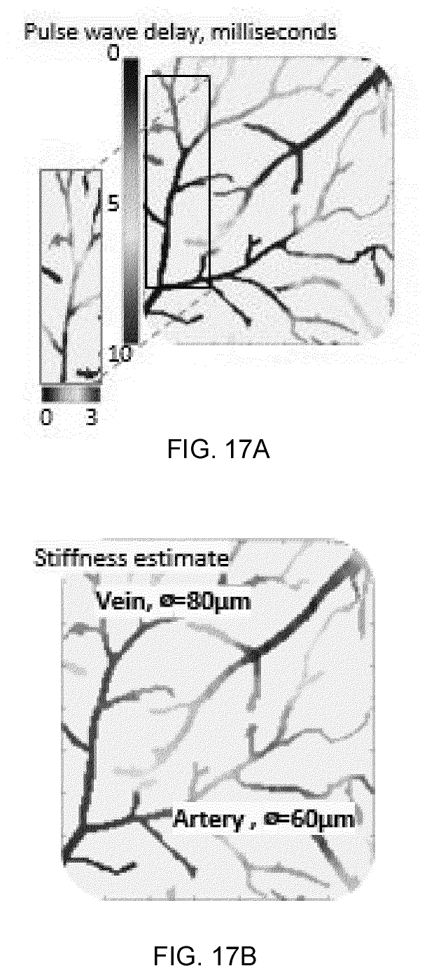

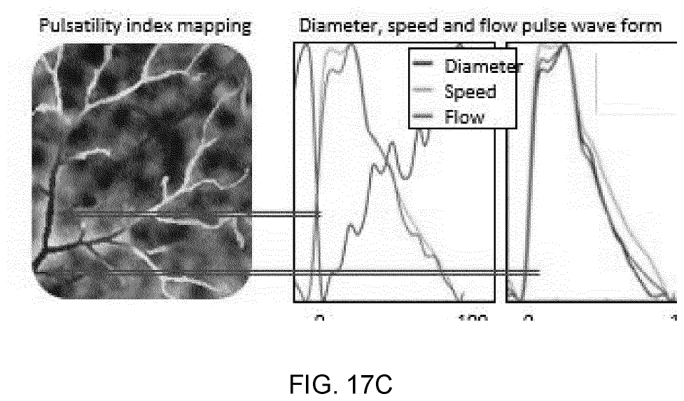

In one embodiment, the presently disclosed method may comprise a main step of Extraction. The main step of Extraction, as shown in FIG. 16A, may comprise the steps of: based on low-noise quantitative time-stamp based average blood flow index and a vessel mask, measuring a foot-to-foot pulse wave delay (FIG. 17A) between nodes of each vessel (608A);—) calculating per vessel pulse wave velocity (PWV); and—) calculating microcirculatory stiffness (FIG. 17B and FIG. 16A, step 608B).

In one embodiment, the main step of Extraction may comprise the steps of: based on low-noise time-stamp based average blood flow index and a vessel mask, measuring a foot-to-foot pulse wave delay between nodes of each vessel; calculating per vessel pulse wave velocity (PWV); and calculating microcirculatory stiffness.

In particular, use of quantitative blood flow index may result in higher accuracy for pulsativity, resistive indexes and for flow, as compared to use of blood flow index that has not undergone quantification step.

As the vessel mask provides the spatial coordinates of the nodes of the vessels, the inventors have realized that a low noise quantitative time-stamp based average blood flow index may be used in combination with the vessel mask to measure a foot-to-foot pulse wave delay, that is a pulse wave delay (FIG. 17A) between nodes of the vessels. Knowing the length of the vessels a pulse wave velocity may be calculated and, based on the pulse wave velocity, stiffness (FIG. 17B) of vessels may be extracted. In this context, it is understood by length of the vessel to the distance along a selected vessel, between the points used to measure the pulse wave velocity. For example, a vessel length might comprise nodes of vessels, wherein a bifurcation of nodes occur. In the case of FIG. 17A, the length of the vessel is described in the inset of the image in the left hand side of the figure. Said length comprises five nodes wherein the measured vessel bifurcates, along the length vessel between the two points used to calculate the pulse wave velocity (the top part and the bottom part of the vessel in the inset image).

In one embodiment of the present disclosure, the inventors have further realized that the low-noise quantitative time-stamp based average blood flow index may be used to further calculate a vascular conducted response (FIG. 16B, 608C).

In one embodiment of the presently disclosed method, based on low-noise quantitative time-stamp based average blood flow index, pulsatility index, resistance index, diameter, flow, vasomotion may be extracted (FIG. 16B—608C, FIG. 17C).

In one embodiment of the presently disclosed method, based on low-noise time-stamp based average blood flow index, diameter, vasomotion, and vascular conducted response of a vessel may be extracted.

In particular, use of quantitative blood flow index may result in higher accuracy measurement of biomarkers, as compared to use of blood flow index that has not undergone quantification step.

### Output Biomarkers

In one embodiment of the present disclosure, the processing unit is configured to output at least one of the calculated features/biomarkers (609).

In one embodiment of the present disclosure the system may comprise a display for displaying at least one of the calculated features.

It has to be understood that the presently disclosed system may be configured to execute all and/or any one of the steps of the presently disclosed method. It is also understood that all or any one of the steps of the presently disclosed method may be carried out by the presently disclosed system.

### EXAMPLE

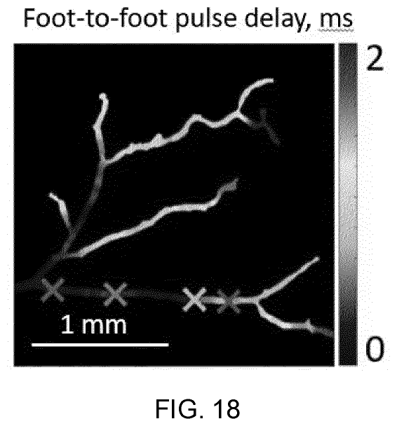

FIG. 18 shows experimental data obtained performing the presently disclosed method. Pulse wave velocity measurements with High-Speed Laser Speckle Contrast Imaging are performed. A camera of a framerate of 5000 frames per second is used to monitor the pulse wave propagation on the studied middle cerebral artery of a control mouse generated by the heartbeat. The color-scale bar shows the map of the foot-to-foot pulse wave delay.

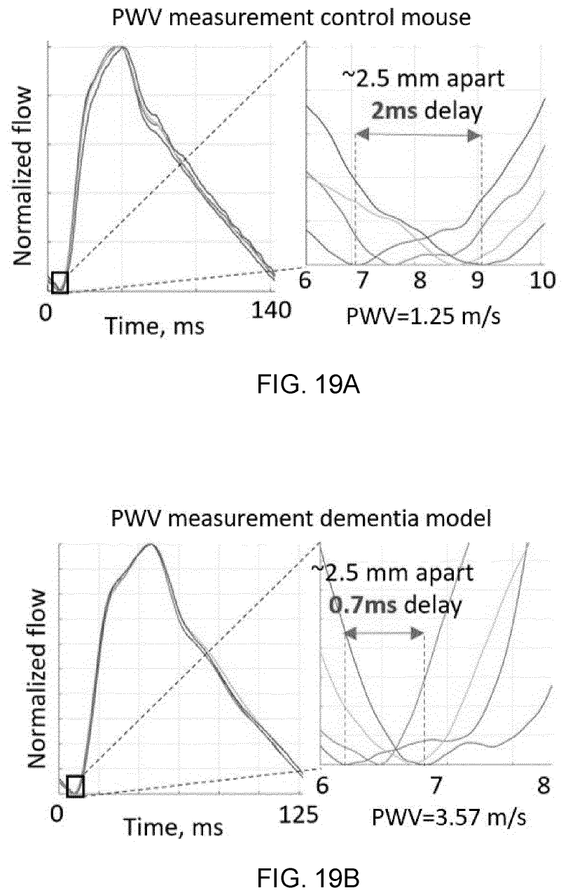

FIG. 19A shows the normalized blood flow (pulse wave form) measured in the locations marked with crosses in FIG. 18. Accordingly, the colour of the lines shown in FIG. 19A correspond to the colour of the crosses in FIG. 18. It is measured a 2 ms delay for a 2.5 mm distance between measurements, indicating a pulse wave velocity of 1.25 m/s.

FIG. 19B shows the normalized blood flow measured in a comparable vessel from FIG. 18 and FIG. 19A in a mouse model of Alzheimer's disease. The measured pulse wave form is 3.57 m/s.

The results obtained in FIG. 19A and FIG. 19B might be used for the calculation of relevant variables related to the health of the imaged vessels, such as for example vessel stiffness, with a higher accuracy than prior art techniques due to the unprecedented precision in the measurement of the extracted data.

The inventors are familiar with the prior art scientific article Patel, Dwani D., et al., Translational Vision Science & Technology 10.9 (2021): 19-19, 2021. In said article, the authors specialized the obtained results on animals while performing state of the art laser speckle contrast imaging. The article does not show results on pulse wave velocity due to the limited used framerate camera of ˜100 fps. Hence, no stiffness can be extracted from the measured vessels. In addition, pulse wave delay in Patel et al. is measured between arterial and venular sides, implying that the distance along the vascular tree between the measured two points is unknown and cannot be estimated. Therefore, it is not possible to measure pulse wave velocity in Patel et al., since measuring pulse wave velocity requires that both the pulse delay and the distance are known.

In contrast, it is object of the present disclosure the measurement of pulse wave velocity and the calculation of the vessel stiffness of animals, making it mandatory the use of high framerate cameras of at least ˜1000 fps, as described in the previous sections. Said high framerate camera must take into account the maximum radiation exposure that the tissue can be exposed to, as higher framerates require higher exposure due to the shorter integration time of each frame, among other considerations described in the previous sections.

Additionally, a laser source may be any highly coherent laser source. Typically, near-infra-red laser sources are used in the field due to the low interaction with the vessels of the used wavelength.

### Further Details

1. A high speed laser speckle contrast imaging system for characterizing pressure or pulse wave propagation or vascular conducted response in at least one vessel of a biological target, the apparatus comprising:
   a laser source generating a laser radiation;
   a high-speed camera configured for capturing at least 1000 frames per second (fps), preferably at least 5000 fps, more preferably 6000 fps of the target;
   an optical sub-system configured for 1) guiding the laser radiation from the laser source to the target 2) and for collecting and guiding a back-scattered light from the target to the camera;
   a processing unit configured for receiving and processing raw image data from the camera for calculating at least one feature related to the pressure or pulse wave propagation or vascular conducted response.
2. The high-speed laser speckle contrast imaging system according to item 1, wherein the at least one vessel is at least one microcirculatory vessel.
3. The high-speed laser speckle contrast imaging system according to any one of the preceding items, wherein the target is a retina of a human or animal eye.
4. The high-speed laser speckle contrast imaging system according to any one of the preceding items, wherein the at least one feature related to the pressure wave propagation is at least a pulse wave velocity in the at least one vessel.
5. The high-speed laser speckle contrast imaging system according to item 4, wherein the processing unit is further configured to extract stiffness of the vessel based on pulse wave velocity.
6. The high-speed laser speckle contrast imaging system according to any one of the preceding items, wherein the at least one feature is at least a vascular conducted response in the at least one vessel.
7. The high-speed laser speckle contrast imaging system according to any one of the preceding items, wherein the laser source is a Near-Infra-Red (NIR) laser source.
8. The high-speed laser speckle contrast imaging system according to any one of the preceding items, wherein, the vessels are microcirculatory blood vessels of diameter size preferably less than 100 micrometers, more preferably less than 80 micrometers in a retina of a human or animal eye.
9. The high-speed laser speckle contrast imaging system according to any one of the preceding items, wherein the vessels have a length above or equal to 0.2 mm, or preferably comprised between 0.2 and 10 mm, more preferably 0.2 to 2 mm, and a pulse wave velocity is up to 7 m/s for a vessel of length of 1.5 mm.
10. The high-speed laser speckle contrast imaging system according to any one of the preceding items, wherein the camera is a high sensitivity CMOS camera, having a sensitivity of at least substantially 16000 ISO, preferably at least 32000 ISO, more preferably at least 64000 ISO and/or at least 50% quantum efficiency, preferably at least 60% quantum efficiency, more preferably at least 70% quantum efficiency at a near infra-red spectrum, and wherein said camera has a pixel size of at least 5 micrometres, preferably at least 20 micrometers, and wherein said camera has a near to zero delay between frames, that is the delay is negligible compared to an exposure time of the camera.
11. The high-speed laser speckle contrast imaging system according to any one of the preceding items, wherein the camera has a pixel size such that a speckle to pixel size ratio is below 2, preferably below 1, even more preferably 0.5.
12. The high-speed laser speckle contrast imaging system according to any one of the preceding items, wherein the optical sub-system comprises a mirror with a pinhole, or a polarizing beam splitter or a 9:1 or higher ratio beam splitter.
13. The high-speed laser speckle contrast imaging system according to any one of the preceding items, wherein the optical sub-system has an at least 90% transmission, preferably at least 95% transmission, more preferably a nearly 100% transmission.
14. A high-speed laser speckle contrast imaging method for characterizing pressure wave or pulse wave propagation or a vascular conducted response in at least one vessel of a biological target, the method comprising the steps of:
   irradiating the target by laser radiation;
   capturing at least 1000 frames per second (fps), preferably at least 5000 fps, more preferably 6000 fps of the target by a high-speed camera;
   calculating at least one feature related to the pressure wave or pulse wave propagation or the vascular conducted response, based on raw image data from the camera, using a processing unit.
15. The method according to item 14, wherein the laser irradiation on the target is below 1 mW/cm2, preferably substantially below 0.25 mW/cm2, such that the irradiation is below a maximum permissible exposure for the target.
16. The method according to item 14, wherein the target is a portion of a retina or a retina of a human or animal eye, wherein the calculated feature is a pulse wave velocity and/or a stiffness of at least one vessel in the retina or a vascular conducted response in a vessel, and wherein the method is further comprising the step of, based on raw image data, calculating spatial contrast frames.
17. The method according to item 14 or 16, further comprising the step of applying motion registration to raw image data.
18. The method according to any one of items 16-17, further comprising the steps of calculating accurate time-stamp based average contrast frames by averaging contrast frames belonging to a same time-stamp cycle, such as a same phase of a heart beat or calculating time-stamp based temporal contrast frames from raw data belonging to a same time-stamp cycle.
19. The method according to any one of items 16-18, further comprising the steps of: based on spatial contrast, calculating spatially averaged contrast time series; identifying timestamps of positive and negative peaks of said time series; discarding peaks affected by motion/noise artifacts; and calculating time-stamp based average contrast frames by averaging contrast frames belonging to a same time-stamp cycle, such as a same phase of a heartbeat.
20. The method according to any one of items 16-19, further comprising the steps of: based on spatial contrast, calculating spatially averaged contrast time series; identifying timestamps of positive and negative peaks of said time series; discarding peaks affected by motion/noise artifacts; and calculating time-stamp based temporal contrast frames from raw data belonging to a same time-stamp cycle, such as a same phase of a heartbeat.
21. The method according to item 20, wherein phases of a heart beat are identified by analysing peaks of time series, and/or by correlating with data produced by an electrocardiogram (ECG), and wherein the method further comprises the step of interpolating the time series to scale heart beats and remove heart rate short term variability effects.
22. The method according to any one of items 16-21, further comprising the step of obtaining nodes for all vessels in the target by segmentation and skeletonization of temporal contrast frames for different surrogate exposure times.
23. The method according to any one of items 16-22, further comprising the steps of: calculating temporal contrast frames over all multi-exposure surrogate frames for different surrogate exposure times; segmenting the temporal contrast frames to get vascular structure masks or vessels masks; combining masks to create a final mask for the vessels segmentation; skeletonizing the final mask; and finding nodes for all vessels.
24. The method according to any one of items 16-23, further comprising the steps of calculating dynamic (ρ) and/or static scattering component, dynamics regime (n) and offset (C), based on fitting average spatial contrast frames obtained from multi-exposure surrogate frames for different surrogate exposure times with up to 3 light scattering models, and calculating a quantitative blood flow index as a function of time and space coordinates or a quantitative time-stamp based average blood flow index, using spatial contrast frames or time-stamp based average contrast frames and fitted parameters.
25. The method according to any one of items 16-24, further comprising the steps of: generating multi-exposure surrogate frames from registered raw image data; calculating average spatial contrast frames based on multi-exposure surrogate frames for different surrogate exposure times; fitting the average spatial contrast frames with at least one and up to 3 light scattering models; calculating fitted parameters dynamic and/or static scattering component (ρ), dynamics regime (n) and offset (C); calculating a quantitative blood flow index as a function of time and space coordinates or a quantitative time-stamp based average blood flow index, using spatial contrast frames or time-stamp based average contrast frames and fitted parameters.
26. The method according to item 25, wherein the light scattering models are at least one in the following selection: multiple scattering ordered motion or single scattering unordered motion, multiple scattering unordered motion, single scattering ordered motion.
27. The method according to item 22, 23, 25, wherein multi-exposure surrogate frames are obtained by applying moving average filters of variable length, and wherein each multi-exposure surrogate frame may be obtained from one or more frames of the raw image data.
28. The method according to any one of item 16-27, further comprising the steps of: based on quantitative time-stamp based average blood flow index and based on a final vessel mask, calculating a low-noise quantitative time-stamp based average blood flow index obtained by segmentation-based spatial averaging of quantitative time-stamp based average blood flow index, such that dynamics of different vessels are not mixed with each other.
29. The method according to any one of items 16-28, further comprising the steps of: based on time-stamp based average blood flow index and based on a final vessel mask, calculating a low-noise time-stamp based average blood flow index obtained by segmentation-based spatial averaging of time-stamp based blood flow index, such that dynamics of different vessels are not mixed with each other.
30. The method according to any one of items 16-29, further comprising the steps of: based on time-stamp based contrast frames and based on a final vessel mask, calculating low-noise time-stamp based average contrast frames obtained by segmentation-based spatial averaging of time-stamp based average contrast frames, such that dynamics of different vessels are not mixed with each other.
31. The method according to any one of items 16-30, further comprising the steps of: based on low-noise quantitative time-stamp average based blood flow index and a vessel mask, measuring a foot-to-foot pulse wave delay between nodes of each vessel; calculating per vessel pulse wave velocity (PWV); and—) calculating microcirculatory stiffness.
32. The method according to any one of items 16-31, further comprising the steps of: based on low-noise time-stamp average based blood flow index and a vessel mask, measuring a foot-to-foot pulse wave delay between nodes of each vessel; calculating per vessel pulse wave velocity (PWV); and calculating microcirculatory stiffness.
33. The method according to any one of items 16-32, further comprising the steps of: based on low-noise quantitative time-stamp based average blood flow index, extracting pulsatility index, resistance index, diameter, flow, vasomotion, and calculating vascular conducted response of a vessel.
34. The method according to any one of items 16-33, further comprising the steps of: based on low-noise time-stamp based average blood flow index, extracting diameter, vasomotion, and calculating vascular conducted response of a vessel.
35. The system of any of preceding items 1-13, configured to execute the steps according to any one of items 14-34.

1. A high speed laser speckle contrast imaging system for characterizing pressure wave propagation or pulse wave velocity of one vessel of a biological target, the apparatus comprising:
a laser source for generating laser radiation;
a high-speed camera configured for capturing at least 1000 frames per second (fps);
an optical sub-system configured for 1) guiding the laser radiation from the laser source to the target 2) and for collecting and guiding a back-scattered light from the target to the camera:
a processing unit configured for receiving and processing raw image data from the camera for calculating the pressure wave propagation or pulse wave velocity over a length of said vessel.

2. The high-speed laser speckle contrast imaging system according to claim 1, wherein the vessel is a microcirculatory vessel and wherein the target is a retina of a human or animal eye.

3. The high-speed laser speckle contrast imaging system according to claim 1, wherein the high-speed camera is configured for capturing at least 5000 fps or at least 6000 fps of the target.

4. The high-speed laser speckle contrast imaging system according to claim 1, wherein said length of the vessel is less than 1 mm, or less than 0.2 mm or less than 0.16 mm.

5. The high-speed laser speckle contrast imaging system according to claim 1, wherein the processing unit is further configured to extract stiffness of the vessel based on the pulse wave velocity.

6. The high-speed laser speckle contrast imaging system according to claim 1, wherein the laser source is a highly coherent laser source.

7. The high-speed laser speckle contrast imaging system according to claim 1, wherein the vessel is as microcirculatory blood vessel of diameter size less than 100 micrometers, in a retina of a human or animal eye.

8. The high-speed laser speckle contrast imaging system according to claim 1, wherein the camera is a high sensitivity CMOS camera, having a sensitivity of at least 16000 ISO, and at least 50% quantum efficiency, at a near infra-red spectrum, and wherein said camera has a pixel size of at least 5 micrometres and wherein said camera has a near to zero delay between frames, such that the delay is negligible compared to an exposure time of the camera, and wherein the camera has a pixel size such that a speckle to pixel size ratio is below 2.

9. The high-speed laser speckle contrast imaging system according claim 1, wherein the optical sub-system comprises a mirror with a pinhole, or a polarizing beam splitter or a 9:1 or higher ratio beam splitter.

10. The high-speed laser speckle contrast imaging system according to claim 1, wherein the optical sub-system has a nearly 100% transmission.

11. A high-speed laser speckle contrast imaging method for characterizing pressure wave or pulse wave velocity in a vessel of a biological target, the method comprising the steps of:
irradiating the target by laser radiation;
capturing at least 1000 frames per second (fps), of the target by a high-speed camera;
calculating the pressure wave propagation or pulse wave propagation over a length of said vessel, based on raw image data from the camera, using a processing unit.

12. The method according to claim 11, wherein said length of the vessel is less than 1 mm, or less than 0.2 mm or less than 0.16 mm.

13. The method according to claim 12, wherein the laser irradiation on the target is below 1 mW/cm2, or below 0.25 mW/cm2, such that the irradiation is below a maximum permissible exposure for the target.

14. The method according to claim 13, wherein the target is a portion of a retina or a retina of a human or animal eye, wherein a stiffness of said vessel is calculated based on the pulse wave velocity, and wherein the method is further comprising the step of, based on raw image data, calculating spatial contrast frames.

15. The method according to claim 14, further comprising the step of calculating accurate time-stamp based average contrast frames by averaging contrast frames belonging to a same time-stamp cycle, or a same phase, of a heart beat or calculating time-stamp based temporal contrast frames from raw data belonging to a same time-stamp cycle.

16. The method according to claim 11, further comprising the step of obtaining nodes for one or more vessels in the target by segmentation and skeletonization of temporal contrast frames for different surrogate exposure times.

17. The method according to claim 11, further comprising the steps of calculating dynamic (ρ) and/or static scattering component, dynamics regime (n) and offset (C), based on fitting average spatial contrast frames obtained from multi-exposure surrogate frames for different surrogate exposure times with up to 3 light scattering models, and calculating a quantitative blood flow index as a function of time and space coordinates or a quantitative time-stamp based average blood flow index, using spatial contrast frames or time-stamp based average contrast frames and fitted parameters, wherein the light scattering models are at least one in the following selection: multiple scattering ordered motion or single scattering unordered motion, multiple scattering unordered motion, single scattering ordered motion.

18. The method according to claim 11, further comprising the steps of: based on quantitative time-stamp based average blood flow index and based on a final vessel mask, calculating a low-noise quantitative time-stamp based average blood flow index obtained by segmentation-based spatial averaging of quantitative time-stamp based average blood flow index, such that dynamics of different vessels are not mixed with each other.

19. The method according to claim 11, further comprising the steps of: based on low-noise quantitative time-stamp average based blood flow index and a vessel mask, measure ng a foot-to-foot pulse wave delay between nodes of each vessel; calculating per vessel pulse wave velocity (PWV); and calculating microcirculatory stiffness.

20. The system of claim 1, configured to execute any one of the following steps:
irradiating the target by laser radiation;
capturing at least 1000 frames per second (fps), of the target by a high-speed camera; and
calculating the pressure wave propagation or pulse wave propagation over a length of said vessel, based on raw image data from the camera, using a processing unit.

21. The method according to claim 11, wherein at least 5000 fps, or at least 6000 fps, of the target are captured by a high-speed camera.

22. The high-speed laser speckle contrast imaging system according to claim 6, wherein the laser source is a Near-Infra-Red (NIR) laser source.
